(19) United States
(12) Patent Application Publication
(10) Pub. No.: US 2010/0279319 A1
(43) Pub. Date: Nov. 4, 2010

(54) Detection of plasmodium falciparum histidine-rich protein II in saliva malaria patients

(76) Inventor: Jonathan K. Stiles, Powder Springs, GA (US)

Correspondence Address: MORRIS MANNING MARTIN LLP, 3343 PEACHTREE ROAD, NE, 1600 ATLANTA FINANCIAL CENTER, ATLANTA, GA, 30326, US

(21) Appl. No.: 12/453,079

(22) Filed: Apr. 29, 2009

(51) Int. Cl. G01N 33/569

(52) U.S. Cl. 435/7.22

(57) ABSTRACT

The detection of PfHRP II in saliva offers a practical, cost-effective alternative to PfHRP II detection in blood as a means for diagnosis of malaria. Collection of saliva is non-invasive, simple, safe, stress free, painless and can be accomplished in primitive settings. The use of Malaria Antigen ELISA kits (CELISA, Cellabs, Australia) used in accord with known procedures for testing blood samples.

This discovery was supported by the government of the United States of America through its agencies. Supporting grants were RR03034 from NIH/NCRR/RCMI and R21 TW006804-01 from NIH-FIC. Hence, certain rights of the United States government apply.

### FIELD AND BACKGROUND OF THE INVENTION

This invention relates to detection of Plasmodium falciparum Histidine-rich Protein II (HPR II) antigen in Saliva of Malaria Patients as means of diagnosing malaria.

Malaria transmission and mortality rates remain unchanged in endemic countries lacking adequate health care and malaria control despite the use of preventive measures and treatments against malaria. A major obstacle to effective malaria control is the lack of affordable and accurate malaria diagnostics and treatment, which has led to misuse and abuse of anti-malarial drugs and the development of drug resistant parasites.

Microscopic examination of blood smears, the conventional method for P. falciparum detection, is currently being augmented with antigen- and PCR-based rapid diagnostic tests (RDTs) for blood. However, inaccurate microscopic evaluation of blood smears has resulted in misdiagnoses and misclassification of malaria severity. Blood taboos and increased risk of accidental infections due to needle pricks continue to impact malaria diagnosis negatively. In nonspecialized laboratories microscopic evaluation of blood smears is slow and may lead to delayed diagnoses and treatment, which contributes to high mortality rates.

Rapid diagnostic tests (RDTs) or “dipstick” tests are currently being used to detect antigens of Plasmodium species in blood or plasma to supplement microscopic evaluation of blood smears to manage tropical febrile disease. The benefits of this approach include rapid turnaround time and ease of use, which allows inexperienced laboratory or clinical staff to make on-the-spot diagnoses in the absence of visible parasites. However, issues associated with cultural objections to the collection of blood in communities with blood taboos and increased risk of needle injuries and disease transmission must be addressed.

Saliva has been used in surveillance of vaccine-preventable diseases, such as measles, mumps, and rubella, and for individual diagnosis of HIV infection by detecting antibodies against the target pathogen. Although P. falciparum HRP II antigen has been detected in erythrocytes, serum, plasma, cerebrospinal fluid, and urine, detection of parasite antigens in saliva of P. falciparum-infected humans has not been reported.

### SUMMARY OF THE INVENTION

The detection of PfHRP II in saliva offers a practical alternative to PfHRP II detection in blood for malaria diagnosis and offers some distinct advantages over blood. Collection of saliva is non-invasive, simple, safe, stress free, painless, and can be done by individuals with limited training, including patients. It does not require blood cell lysis that diminishes HRP II antigen availability and detection. No special equipment is needed for collection and it allows for multiple or serial collections outside of the hospital. Detecting parasite antigens in saliva to determine presence or absence of parasites could be valuable for communities with blood taboos and reduce compliance problems associated with collection of blood. Furthermore, it will provide a cost-effective approach for the screening of large populations in epidemiological surveys while being affordable, rapid, non-invasive, and safe for patients and technicians in resource-poor environments.

### DETAILED DESCRIPTION OF THE INVENTION

It has now been found, surprisingly, that a test used for PfHRP II antigen used to identify the cited antigen in the blood can be used according to methods disclosed below to detect PfHRP II antigen in saliva. This discovery makes it possible to test in settings that were not appropriate when withdrawal of blood from the patient was required.

The studies described herein were conducted at the Korle-Bu Teaching Hospital's Child Health Department, Accra, Ghana, after ethical approval by Morehouse School of Medicine and University of Ghana Medical School.

Basically, the methods of the invention consist of a method of detecting the presence and estimating, by ELISA, the number of Plasmodium falciparum parasites in saliva comprising the steps of collecting a sample of saliva from an individual as described below, exposing the samples of saliva to a support treated with anti-P. falciparum monoclonal capture antibodies, after washing, allowing the support with the saliva to incubate. After appropriate incubation, the plates are usually exposed to a solution which enhances conjugation of the antibodies to P. falciparum and the P. falciparum proteins. Preferably, the plates with the conjugates are then washed before exposure to at least one indicator such as a chromogens or fluorogens which will render the conjugate subject to inspection by visual inspection or by spectrophotometer. The particular antibody used in the kits as provided is an antibody to the PfHRP II. However, other malaria parasite specific proteins may be used.

### Materials and Methods

Malaria Antigen ELISA kits (CELISA, Cellabs, Australia) were used in accord with the instructions provided therewith. This kit measures HRP II production during growth and multiplication of P. falciparum at a specificity of 96% and sensitivity of 98% in whole blood or plasma and can detect P. falciparum parasites at a limit of detection of 0.001%; thus incubation periods with reagents were the same for plasma and saliva for the same patient. The plates provided with the kits are coated with anti-P. falciparum monoclonal capture antibodies. If the P. falciparum antigen is present, it will bind to the coating of the plate.

Saliva was obtained by syringe from the mouths of children who were believed to have been or were known to have been exposed to malaria. In order to obtain sufficient saliva, each child was allowed to chew on a piece of sugar free gum before collection of the sample. However, saliva production may be increased by other means such as by simply exposing the subject from whom saliva is to be obtained for testing to the odor of a well liked food.

Wash Buffer was prepared by adding 50 mL PBS-Tween to 950 mL distilled water. Each kit containing the supplies provided by Cellabs contains positive controls, negative controls, enzyme conjugate, conjugate diluents, substrate chromogen, substrate buffer and stopping solution.

Working strength conjugate is prepared by adding 5 ul conjugate concentrate provided with the kit to 995 ul conjugate diluent.

Working strength substrate is prepared by adding 50 ul of substrate chromogen to 950 ul substrate buffer, then mixing thoroughly. (Stability period is ≦30 minutes)

In accord with the instruction of the kit, 100 ul of the sample, positive control or negative control was pipette into each well. The plates were covered and incubated for 1 hour at room temperature in a humid chamber. (During the last 10 minutes of the incubation period, the working strength conjugate is prepared in order that it be fresh.)

The wells were then washed in accord with the instructions on the kit. 100 ul working conjugate was added to each well and the product was again incubated as above for 1 hour. The wash step is repeated. Prepared fresh working substrate (100 ul) is added to each well. The plates are then incubated in the dark (covered) at room temperature for 15 minutes, after which 50 ul stop solution is added. The results are read visually or in a spectrophotometer. On visual reading, the positive control should be blue before and yellow after stopping. A spectrophotometer can also be used in accord with the teachings of the manufacturer's instruction.

Randomly collected samples (plasma and saliva) from children (22 months to 16 years) reporting to the Child Health Department's diagnostic laboratory were retrospectively analyzed for this study. Malaria positive cases were confirmed by thick film slides. Parasitemia was evaluated on the number of parasites per field (+, 1-10 parasites/100 fields, ++, >10 parasites/100 fields, +++, 1-10 parasites/field, and ++++>10 parasites/field) and at least 100 fields/slide were examined to rule out any negative thick film slide. Thirty thick film positive children and 10 negative children were enrolled. Red blood cells (infected and uninfected) and plasma were separated using Vacutainer Cell Preparation Tubes (CPT) with Sodium Citrate (Becton Dickinson, USA). Saliva was collected in sterile containers and aliquoted into microcentrifuge tubes and stored at −20° C. Saliva samples were centrifuged for 3 min at 14,000 rpm and the supernatants were analyzed by ELISA. Both saliva and plasma samples from the same patient were analyzed on the same plate, date, and conditions for PfHRP II antigen levels. The plasma samples were tested at a 1:2 dilution and all samples were run in duplicates by ELISA according to instructions of the manufacturer. The incubation period for primary and secondary antibodies with the samples was 1 hr each in a humid chamber and 15 min for enzyme development (substrate) in the dark at room temperature. The minimum limit of detection (cut-off level) of the kit was determined according to manufacturer's instructions.

### Results

Of the 30 children testing positive for blood smear, 16 (53%) had detectable PfHRP II antigens in their plasma (Table 1). Thirteen (43%) patients of the 30 positive blood smears were PfRP II positive for saliva samples (Table 1). All patients that were PfHRP II positive for saliva were also positive for plasma. Three patients (P006, P008, and P011) were PfHRP II positive in plasma but negative for saliva samples. Surprisingly, P006 had a mean OD reading (0.144) that is slightly below the cut-off level of 0.161 compared with the other 2 (P008 and P011) PfHRP II negative saliva. This observation suggests that P006 may have PfHRP II in the saliva that is undetectable in the kit used for this study. The 10 negative blood smears were also negative for PfHRP II antigen in both plasma and saliva. In our study the minimum limit of detection (cut-off level) was an OD reading of 0.161, which was determined according to the manufacturer's instructions. In addition, all 13 saliva specimens had lower titers (OD, 0.166-0.427) of PfHRP II with a mean of 0.209±0.07. The sensitivity of PfHRP II detection test for plasma was 53% and 43% for saliva whereas specificity was 100% for both specimens when compared with blood smears.

1-4. (canceled)

5. A method for the diagnosis of malaria in a subject by detecting the presence of Plasmodium falciparum histidine-rich protein II (PfHRPII) in a saliva sample from said subject, comprising:
contacting saliva from said subject with anti-PfHRPII antibody, wherein the antibody is immobilized on a support, and
detecting the binding of PfHRPII to the antibody by ELISA,
wherein the detection of bound PfHRPII from the saliva indicates malaria in said subject.

6. The method of claim 6, wherein said detection is by spectrophotometric means.

7. The method of claim 6, wherein said detection is at a wavelength of 450-650 nanometers.

8. The method of claim 1, wherein said detection is by visual inspection.
